(19) United States
(12) Patent Application Publication
(10) Pub. No.: US 2025/0073169 A1
(43) Pub. Date: Mar. 6, 2025

(54) POLYMER NANOPARTICLES VIA CONDENSED DROPLET POLYMERIZATION

(71) Applicant: CORNELL UNIVERSITY, Ithaca, NY (US)

(72) Inventors: Rong YANG, Ithaca, NY (US); Trevor FRANKLIN, Ithaca, NY (US); Danielle STREEVER, Waleska, GA (US)

(73) Assignee: CORNELL UNIVERSITY, Ithaca, NY (US)

(21) Appl. No.: 18/293,204

(22) PCT Filed: Aug. 2, 2022
(86) PCT No.: PCT/US2022/074425

§ 371 Date: Jan. 29, 2024

Related U.S. Application Data
(60) Provisional application No. 63/228,480, filed on Aug. 2, 2021.
(60) Provisional application No. 63/364,341, filed on May 8, 2022.

(51) Int. Cl. A61K 9/16; A61K 31/131; C08F 212/36; C08F 220/28; C08F 226/06

(52) U.S. Cl. CPC A61K 9/1694; A61K 9/1635; A61K 31/131; C08F 212/36; C08F 220/281; C08F 226/06

(57) ABSTRACT

Provided is a method of synthesizing polymer particles, including: introducing vapor-phase reagents into a reactor having a substrate; forming condensed droplets of the reagents on the substrate; initiating polymerization; and polymerizing the condensed droplets of the reagents, thereby forming polymer particles. Polymer particles are also provided, including those incorporating therapeutic agents.

### CROSS REFERENCE TO RELATED APPLICATIONS

This application claims priority to U.S. provisional application No. 63/228,480, filed on Aug. 2, 2021, and 63/364,341, filed on May 8, 2022. The entire contents of both priority applications are hereby incorporated by reference herein.

### GOVERNMENT LICENSE RIGHTS

This invention was made with government support under DGE-1650441 awarded by the National Science Foundation and CMMI-2144171 awarded by the National Science Foundation. The government has certain rights in the invention.

### BACKGROUND

Polymerization of monomers to form nanoparticles (i.e. bottom-up synthesis) is commonly performed in liquid environments which confers restrictions to monomer chemistries and particle morphologies based on solubility and rheology.

Polymer nanoparticles as nanotheranostic agents have chemical versatility that supports a variety of bioresponsive behaviors aimed at diagnosis and treatment of diseases. Conventional bottom-up synthetic techniques such as emulsion polymerization take place in a liquid environment that confers certain restrictions on the resulting particles. These include: shape, as solutions/emulsions primarily produce spherical particles, yet shape can alter in vivo biodistribution; chemical compatibility, as solutions/emulsions tolerate molecules of specific solubilities, thus some monomers and other therapeutic molecules can be restricted by a choice of solvent; and efficiency, as many nanoparticle synthetic protocols, especially those for non-spherical particles like domes, take hours or days to complete.

Thus, a need exists for improved polymer particles, including those comprising a therapeutic agent, and for methods of making the same.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

In this application, where a document, act or item of knowledge is referred to or discussed, this reference or discussion is not an admission that the document, act or item of knowledge or any combination thereof was, at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

Briefly, embodiments of the present invention present a synthetic technique (e.g., an all-dry synthetic technique) to produce polymer nanoparticles (e.g., dome-shaped polymer nanoparticles) named Condensed Droplet Polymerization (CDP). CDP is a rapid, versatile, solvent-free technique for the bottom-up synthesis of polymer nanoparticles in a vapor deposition apparatus. Without solubility constraints of solution-based syntheses and by leveraging vapor phase initiation, CDP enables the synthesis of hemispherical polymer nanoparticles from a wide range of monomer types with diameters across the full nanoscale range. Various embodiments of this technique leverage a chemical vapor deposition (CVD) apparatus to deliver all reagents in the vapor phase, thereby avoiding solubility restrictions. In various embodiments, CDP produces non-spherical polymer nanoparticles having a wide range of chemistries and polymerization is complete within seconds.

In some embodiments, the present invention solves the problem(s) of (i) synthesizing polymer nanoparticles (or particles larger than nano-scale) in a bottom-up fashion with no solution-based steps needed to bolster the versatility of products by removing solubility constraints; (ii) incorporating an additional agent (e.g., a therapeutic agent, such as a chemotherapy drug), into the particle without using solvents in order to avoid restrictions based on solubility of both polymerization reagents and therapeutic agents; and/or (iii) achieving programmable pharmacokinetics for the encapsulated therapeutic via precise control of the particle structure and size, which could enable precise and personalized medicine.

While some polymer particles containing therapeutics are known, and polymer thin films have been used to encapsulate therapeutics (see, e.g., Decandia G, Palumbo F, Treglia A, et al. Initiated Chemical Vapor Deposition of Crosslinked Organic Coatings for Controlling Gentamicin Delivery, Pharmaceutics. 2020; 12(3):213. Published 2020 Mar. 1. doi: 10.3390/pharmaceutics 12030213), embodiments of the present invention provide advantages over the state of the art. For example, prior to the present invention, solvent-free synthesis of nanoparticles containing therapeutics has not been achieved. The commercialization of drug-encapsulated polymer nanoparticle synthesis has been limited by the bottleneck issue of residual solvents and their potential toxicity. The present disclosure enables the complete removal of solvents, thereby promising an accelerated path to deployment in nanomedicine, especially in oncology where non-polar therapeutics require the use of toxic and non-polar solvents in existing synthesis technologies. Further, prior art liquid-based synthetic techniques create spherical particles, but the present invention is able to provide dome-shaped and even porous particles. Further, other technologies chemically bond therapeutic agents to the surface of the particles, absorb them into particles like a sponge, or encapsulate a drug core in a polymer shell. According to embodiments of the present invention, on the other hand, the therapeutic is incorporated during the synthesis and, in some embodiments, is equally distributed throughout without soaking to absorb. Further, according to embodiments of the present invention, size of the particles and distribution of any additional agent (e.g., therapeutic agent) within the particle, hence their release kinetics, can be controlled precisely without needing to change the synthesis procedure.

In a first aspect, the invention provides a method of synthesizing polymer particles, said method comprising:
introducing vapor-phase reagents into a reactor having a substrate;
forming condensed droplets of the reagents on the substrate;
initiating polymerization (e.g., by introducing polymerization initiator into the reactor or via photoinitiation); and
polymerizing the condensed droplets of the reagents, thereby forming polymer particles.

In a second aspect, the invention provides a polymer particle comprising:
polymer material; and
optionally, mixed with the polymer material, an additional agent (e.g., a therapeutic agent).

### BRIEF DESCRIPTION OF THE DRAWINGS

FIG. 1 shows an example of a condensed droplet polymerization (CDP) protocol that utilizes an initiated chemical vapor deposition (iCVD) reactor to synthesize solid PNPs from vapor phase reagents.

FIG. 2 depicts a CDP reactor schematic. An adapted iCVD reactor is depicted with components for CDP, including a thermoelectric cooler (TEC) module (light gray) on which the prepared substrate sits and a high-magnification camera (top center).

FIG. 3 depicts SEM images that show the surface roughness of a poly(1H,1H,2H,2H-perfluorodecyl acrylate) (PPFDA) thin film deposited by initiated chemical vapor deposition (iCVD) in its as-deposited, rough morphology (left) and after heating (right) when the surface has flattened.

FIG. 4 depicts embodiments of PNPs synthesized by the CDP technique. SEM images show nanoparticles from the polymerization of droplets from 3 monomers: HEMA, DVB, and 4VP. PNPs of the same chemistry are grouped by row. Particles with diameters spanning the full nanoscale range are grouped by column.

FIGS. 5A-C show spectra relating to the chemical composition and size dispersity of PHEMA nanoparticles. FIG. 5A is FTIR spectra of a PHEMA thin film representing polymerized HEMA, the PPFDA substrate layer, and the same base layer with PHEMA nanoparticles synthesized on top. Gray background highlights the peaks associated with PHEMA that are not present in the base layer and visible after the synthesis of the PHEMA nanoparticles. FIG. 5B shows an SEM image of PHEMA particles (top left) with energy dispersive x-ray spectroscopy (EDX) mapping of fluorine (top right), carbon (bottom left), and oxygen (bottom right). FIG. 5C is a histogram of PHEMA nanoparticle diameters representing 416 particles analyzed from SEM images taken at 10 locations across the substrate.

FIG. 6 is an SEM showing side and bottom views of an embodiment of PHEMA nanoparticles. SEM imaging from a tilted sample shows the hemispherical profile of the polymer nanoparticles synthesized by CDP. An upturned particle illustrates that the particles are fully solidified, rather than a hollow shell.

FIGS. 7A-C show results of programming polymer nanoparticle (PNP) embodiment particle shape through contact angle. FIG. 7A shows that alteration of the substrate surface energy changes the contact angle (θ_{CA}) of the liquid monomer droplet upon condensation. FIG. 7B shows contact angle of each liquid monomer (standard script) on a PPFDA or PDVB coated substrate (subscript) (n=5 for all monomers on PPFDA; n=4 for HEMA on PDVB). SEM images confirm the side angle profile of the nanoparticles resulting from each monomer/substrate pair. FIG. 7C shows height to weight (diameter) ratio (left y-axis) of nanoparticles polymerized from the same monomers and substrates of FIG. 7B measured by AFM tracing of the particles (n=4). The right y-axis shows the height of the individual particles, all of which have a similar diameter. All bars represent the mean and error bars represent the standard deviation.

FIG. 8 is a contact angle goniometry image of 2-hydroxyethyl methacrylate on a fluorinated base layer applied directly to a substrate.

FIG. 9 depicts Scheme 1, by which embodiments of polymer particles comprising therapeutics were prepared.

FIG. 10 shows SEM images of resultant particles of poly(2-hydroxyethyl methacrylate) containing the chemotherapy drug chlormethine.

FIG. 11 depicts an SEM image and corresponding point-EDX scan showing chlorine atoms verifying the presence of chlormethine and oxygen showing the presence of HEMA in embodiments of inventive therapeutic-incorporating particles.

### DETAILED DESCRIPTION

In the following and attached description, reference is made to the accompanying drawings and text that form a part hereof, and in which is shown by way of illustration specific embodiments which may be practiced. These embodiments are described in detail to enable those skilled in the art to practice the invention, and it is to be understood that other embodiments may be utilized and that structural, logical and electrical changes may be made without departing from the scope of the present invention. The following and attached description of example embodiments is, therefore, not to be taken in a limited sense, and the scope of the present invention is defined by the appended claims.

Polymer nanoparticles (PNPs) are valuable materials in a range of applications, including many that are biomedical in nature. Within biomedical research, PNPs are commonly studied as vessels for drug and gene therapy delivery. As therapeutic delivery systems, polymeric nanoparticles benefit from amenability to chemical modification, structural and chemical features that enable targeted delivery, and biocompatibility. Clinical trials for these non-invasive treatments have resulted in Food and Drug Administration (FDA) or European Medicines Agency (EMA) approval of over 25 nanoparticles-based medicines and over 75 ongoing clinical trials have been identified including polymeric nanoparticles targeting breast, lung, prostate, head, neck, cervical, bladder, pancreatic and biliary cancers. The value of PNPs extends beyond biomedical applications into fields such as agriculture and food science. Polymeric nanoparticles have shown to be effective delivery tools for plant growth regulators to improve fruit production in plants. Polymeric particle matrices have been tested as delivery agents for flavor enhancers, antioxidants, bioactive compounds, and prebiotics, as well as adsorbents of chemical residues in the food sector. PNPs are also valuable platforms onto which expensive metals can be applied to generate cheaper, high surface area catalysts for chemical synthesis and blends with inorganic compounds are useful for optoelectronic properties. Non-spherical nanoparticles have also been investigated for a range of biomedical applications in which shape impacts biodistribution and rates of cellular uptake.

In particular, bottom-up PNP synthesis involves an input of monomers which are polymerized directly into a nanoparticle shape, a process conventionally involving solution-based steps in a liquid environment. The most common bottom-up method is emulsion polymerization in which a monomer with low water solubility is introduced to water along with a water-soluble initiator and surfactant. Other bottom-up synthetic techniques include membrane emulsification and interfacial polymerization in which step polymerization occurs at the interface of two phases in which two reactive monomers are dissolved. Hemispherical polymer nanoparticles, in particular, have been achieved by the cleavage of Janus particles formed in solution and may be studied for self-assembly, enhancement of mechanical properties, or as nano-lenses. Utilization of solvents, which are often toxic, introduces rheological influence on particle shape and results in impurities that require costly additional removal steps for a purified product. The need for purification elongates the already lengthy synthetic protocols that are typically on the order of hours. An ideal remedy to these challenges is a rapid technique that can produce a pure product in one step without dissolving or dispersing monomer(s) or reagents in solution. A synthetic approach that introduces only monomers and initiator to a solution-free system would evade the requirement of purification steps and restrictions on access to monomers based on solubility.

Some vapor deposition techniques characteristically avoid solution-based procedures using only reagents in the vapor phase, but are more commonly used to produce polymer thin films on a substrate. Vapor deposition has been used to synthesize inorganic nanoparticles and to modify or encapsulate nanoparticles synthesized by conventional methods, but has not been applied to the synthesis of solid, template-free polymer particles without involving a liquid substrate. To the best of the Applicant's knowledge, there is only one liquid-free nanoparticle production process based on chemical vapor deposition that deposited poly-para-xylylene onto actively sublimating ice droplet templates to form porous nanoparticles. (H.-Y. Tung, Z.-Y. Guan, T.-Y. Liu, H.-Y. Chen, Nat. Commun. 2018, 9, 2564.) Though avoidant of liquid, the porosity of the particles and the restriction to poly-para-xylylenes limits this technology. Instead, a flexible, vapor-based technique is needed that can create PNPs from a range of monomers that yield solid particles.

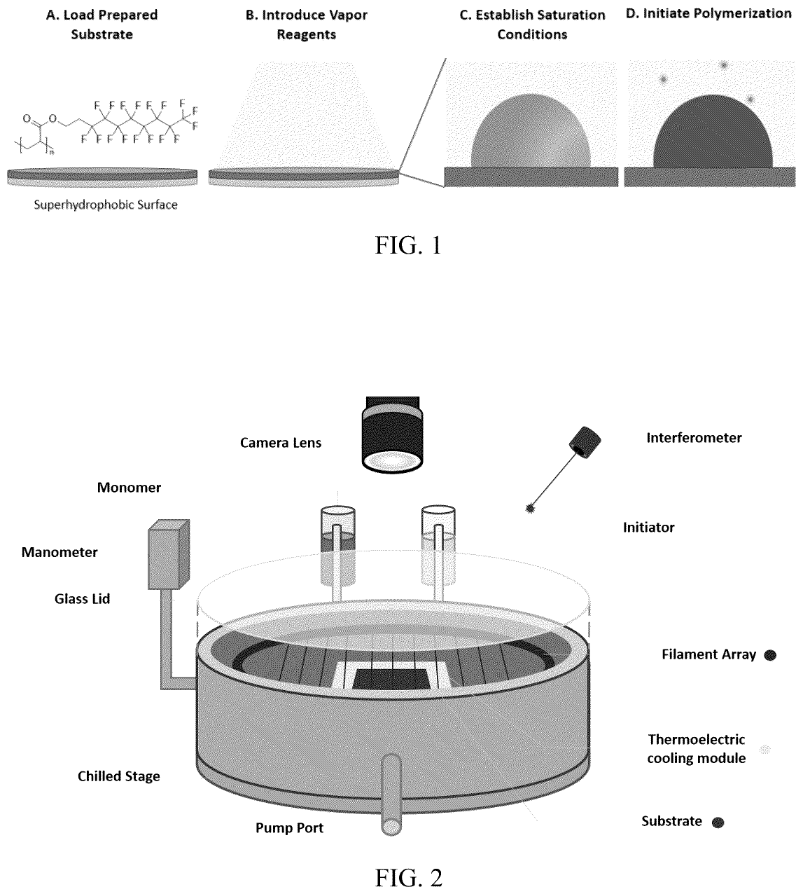

Embodiments of the present invention involve a new technique named condensed droplet polymerization (CDP) that utilizes, in some non-limiting embodiments, an optionally modified initiated chemical vapor deposition (iCVD) reactor to synthesize solid polymer particles (e.g., PNPs) from vapor phase reagents. The depicted CDP protocol, illustrated in FIG. 1, may be described in four steps that can be customized for the monomer(s) of choice, and results in pure, solid, hemispherical polymer particles (e.g., PNPs) atop a flat surface. Briefly, a prepared substrate is loaded (e.g., into an iCVD reactor chamber) and placed under medium vacuum. Said substrate is selected and/or fabricated to feature a low interfacial energy (i.e., high contact angle) between the monomer to be polymerized and the substrate surface. The depicted substrate is covered with a polymer thin film layer (e.g., with perfluorinated side chains). Monomers and other necessary reagents are then metered into the reactor chamber once the chamber is evacuated and isolated from a vacuum pump. With gaseous monomers present, condensation of the monomer onto the substrate surface is forced by increasing the chamber pressure and decreasing the temperature of the substrate to reach the saturation pressure of the monomer. Condensed droplets form at the saturation pressure with a hemispherical shape due to the high contact angle that results from the low interfacial energy. When the droplets are determined to be the desired size, a polymerization initiator is introduced/activated to solidify the condensed droplets in place. In different embodiments of the invention, each step of CDP may be customized to adapt to unique properties of the monomer(s) at hand. Various embodiments yield pure, hemispherical polymer particles (e.g., PNPs) with diameters in the nanometer (nm) or micrometer (μm) range across the substrate surface. Following CDP, polymer particles may be dislodged from the surface and isolated for use in appropriate applications that leverage the unique properties of the functional polymers. Embodiments of the invention thus avoid solubility constraints associated with prior art methods and provide for particles (e.g., nano- and micro-scale particles) comprising a wide range of chemistries.

In a first aspect, the invention provides a method of synthesizing polymer particles, said method comprising:
introducing vapor-phase reagents into a reactor having a substrate;
forming condensed droplets of the reagents on the substrate;
initiating polymerization (e.g., by introducing polymerization initiator into the reactor or via photoinitiation); and
polymerizing the condensed droplets of the reagents, thereby forming polymer particles.

In some embodiments, the vapor-phase reagents include one or more monomers. It is within the purview of a person skilled in the art to be able to select monomers that are amenable to use in the inventive method, and it is contemplated that all such monomers may be used.

In some embodiments, the one or more monomers comprise 2-hydroxyethyl methacrylate, divinylbenzene, 4-vinylpyridine, benzyl methacrylate, ethylene glycol dimethacrylate, 1-vinylimidazole, cyclohexyl methacrylate, 2-(dimethylamino)ethyl methacrylate, or glycidyl methacrylate, or any combination thereof.

In some embodiments, the one or more monomers comprise one or more monomers from the following Table 2.1 from Gleason, K. K. (2015) CVD Polymers: Fabrication of Organic Surfaces and Devices, pages 19-22, or a combination thereof:

TABLE 2
ICVD polymer chemistries.^{a)}

| Polymer class | Monomer/precursor^{b)} | Initiator^{c)} | Reference^{d), e), f)} |
|---|---|---|---|
| Fluorocarbon | HFPO | — | [1], [2], [3], [4], [5], [6], [7], [8], [9], |
| | | | [10], [11] |
| | — | PFBSF | [12], [13], [14], [15], [16], [17], [18] |
| | — | PFOSF | [19] |
| | HFPO + D₃ | — | [20] |
| Organosilicon | [symbol] | — | [21] |
| | [symbol] + HFPO | — | [20] |
| | [symbol] | — | [21], [22] |
| | HVDS | TBPO | [23], [24] |
| | HVDS + V₃D₃ | — | [25], [26] |
| | [symbol] | PFOSF | [14], [27], [28], [29] |
| | — | TBPO | [26], [30], [31], [32] [33], [34], [35], |
| | | | [36], [37] |
| | [symbol] + HVDS | TBPO | [25], [26] |
| | [symbol] | TBPO | [38], [39], [40] |
| Acrylate | AA + NIPAAm + EGDA | TBPO | [41] |
| | DEAAm + EGDA | TBPO | [41] |
| | DEAAm + DMAAm + | TBPO | [41] |
| | DEAEA | TAPO | [42], [43] |
| | DFHA + GMA | TBPO | [44], [45] |
| | DMAAm + MAM + DEGDVE | TBPO | [46], [47] |
| | DMAAm + DEAAm + | TBPO | [41] |
| | DMAAm + NIPAAm + | TBPO | [41] |
| | EA | TAPO | [48] |
| | EA + MAA | TBPO | [49], [50] |
| | MCA | TBPO | [51] |
| | MCA + MAA | TBPO | [51] |
| | nBA | TAPO | [48], [52] |
| | — | TBPO | [38, 53, 54] |
| | nBA + DEGDVE | TBPO | [55] |
| | nHA | TAPO | [48] |
| | nPA | TAPO | [48] |
| | nPrA | TAPO | [48] |
| | NIPAAm | TBPO | [37], [56], [57], [58], [59], [60] |
| | NIPAAm + DEGDVE | TBPO | [56], [57], [58] |
| | NIPAAm + EGDA | TBPO | [41], [61] |
| | NIPAAm + EGDMA | TBPO | [62] |
| | NIPAAm + AA + EGDA | TBPO | [41] |
| | NIPAAm + DMAAm + EGDA | TBPO | [41] |
| | PFDA | TBPO | [37], [40], [54], [59], [63], [64], [65], |
| | | | [66], [67], [68], [69], [70], [71], [72], |
| | | | [73], [74], [75], [76] |
| | PFDA + DVB | TBPO | [72], [75], [77] |
| | PFDA + EGDA | TBPO | [78], [79] |
| | PFDA + HEMA | TBPO | [80], [81], [82], [83], [84] |
| | PFDA + MAA | TBPO | [85], [86] |
| | PrgA | TBPO | [87] |
| | PrgA + EGDA | TBPO | [87] |
| | tBA | TBPO | [88] |
| | tBA + DEGDVE | TBPO | [55], [88], [89] |
| Methacrylate | CHMA | TAPO | [90] |
| | — | TBPO | [91], [92], [93], [94] |
| | — | TBPOB | [94] |
| | CHMA + EGDMA | TAPO | [90] |
| | — | TBPO | [95] |
| | DMAEMA + EGDA | TBPO | [96] |
| | DMAEMA + EGDMA | TBPO | [97], [98] |
| | DPAEMA | TBPO | [99] |
| | FMA | TBPO | [100] |
| | GMA | TAPO | [15], [90], [101], [102] |
| | — | TBPO | [73], [91], [103], [104], [105], [106], |
| | | | [107], [108], [109], [110], [111] |
| | | | [112], [113], [114], [115], [116], |
| | | | [117], [118], [119], [120] |
| | GMA + DFHA | TBPO | [44], [45] |
| | GMA + EGDA | TBPO | [121] |
| | GMA + PFEMA | TBPO | [45] |
| | HEMA | TAPO | [122], [123], [124], [125], [126], |
| | | | [127], [128] |
| | — | TBPO | [38], [53], [54], [59], [66], [68], |
| | | | [129], [130], [131], [132], [133] |
| | HEMA + EGDA | TBPO | [89], [129], [134], [135], [136], [137] |
| | HEMA + EGDMA | TBPO | [138], [139] |
| | HEMA + PFDA | TBPO | [80], [81], [82], [83], [84] |
| | HEMA + PFM | TBPO | [66] |
| | HEMA + PFM + EGDA | TBPO | [140] |
| | HFDMA | TBPO | [141] |
| | MMA | TBPO | [51], [131] |
| | MMA + EA | TBPO | [49], [50] |
| | MMA + EGDA | TBPO | [142] |
| | MMA + EGDMA | TBPO | [49], [50], [143] |
| | MMA + MCA | TBPO | [51] |
| | MMA + PFDA | TBPO | [85], [86] |
| | MMA + PFEMA | TBPO | [44], [45] |
| | MMA | TBPO | [144], [145], [146] |
| | nBMA | TBPO | [54] |
| | neoPMA | TBPO | [147], [148], [149] |
| | neoPMA + EGDA | TBPO | [147] |
| | oNBMA | TMPA | [69], [131], [150] |
| | PFEMA | TBPO | [151], [152] |
| | PFEMA + GMA | TBPO | [45] |
| | PFEMA + MAA | TBPO | [45], [44] |
| | PFM | TBPO | [38], [54], [69], [13] |
| | PFM + EGDA | TBPO | [153] |
| | PFM + HEMA | TBPO | [66] |
| | PFM + HEMA + EGDA | TBPO | [140] |
| | PrgMA | TBPO | [154], [155] |
| Styrene | 4AS | TBPO | [114], [156] |
| | 4AS + MAM | TBPO | [157] |
| | DMAMS | TAPO | [42], [43], [158] |
| | — | TBPO | [159] |
| | S + MAH | TBPO | [160], [161], [162] |
| Other vinyls | 4VP | TBPO | [54], [163], [164], [165], [166] |
| | AVP + DVB | TBPO | [167], [168] |
| | 4VP + EGDMA | TBPO | [76] |
| | mDEB | TBPO | [169] |
| | MAH + 4AS | TBPO | [157] |
| | MAH + DEGDVE | TBPO | [55], [120] |
| | MAH + S | TBPO | [160], [161], [162] |
| | MAH + DMAAm + DEGDVE | TBPO | [46], [47] |
| | MAH + VP + DEGDVE | TBPO | [170] |
| | NVCL | TBPO | [171] |
| | PhAc | TBPO | [172] |
| | VI | TBPO | [173] |
| | VI + VP | TBPO | [173] |
| | VP | TBPO | [173], [174] |
| | VP + EGDA | TBPO | [174] |
| | VP + VI | TBPO | [173] |
| | VP + MAH + DEGDVE | TBPO | [170] |
| Crossslinking | DEGDVE + MAH | TBPO | [55], [170] |
| | DEGDVE + nBA | TBPO | [55] |
| | DEGDVE + NIPAAm | TBPO | [56], [57], [58] |
| | DEGDVE + tBA | TBPO | [55], [88], [89] |
| | DEGDVE + DMAAm + MAH | TBPO | [46], [47] |
| | DEGDVE + MAH + VP | TBPO | [170] |
| | DVB | TBPO | [67], [167], [175] |
| | DVB + 4VP | TBPO | [167], [168] |
| | DVB + PFDA | TBPO | [72], [75], [77] |
| | EGDA | TBPO | [115], [137], [147], [148], [153], |
| | | | [175], [176], [177], [178], [179] |
| | EGDA + DEAAm | TBPO | [41] |
| | EGDA + DMAEMA | TBPO | [96] |
| | EGDA + EGDMA | TBPO | [176] |
| | EGDA + GMA | TBPO | [121] |
| | EGDA + HEMA | TBPO | [89], [129], [134], [135], [136], [137] |
| | EGDA + MAA | TBPO | [142] |
| | EGDA + neoPMA | TBPO | [147] |
| | EGDA + NIPAAm | TBPO | [41], [61] |
| | EGDA + PFDA | TBPO | [78], [79] |
| | EGDA + PFM | TBPO | [153] |
| | EGDA + PrgA | TBPO | [87] |
| | EGDA + VP | TBPO | [174] |
| | EGDA + AA + NIPAAm | TBPO | [41] |
| | EGDA + DMAAm + DEAAm | TBPO | [41] |
| | EGDA + DMAAm + NIPAAm | TBPO | [41] |
| | EGDA + HEMA + PFM | TBPO | [140] |
| | EGDMA | TBPO | [76], [176] |
| | EGDMA + 4VP | TBPO | [76] |
| | EGDMA + CHMA | TAPO | [90] |
| | — | TBPO | [95] |
| | EGDMA + DMAEMA | TBPO | [97], [98] |
| | EGDMA + EGDA | TBPO | [176] |
| | EGDMA + HEMA | TBPO | [138], [139] |
| | EGDMA + MAA | TBPO | [49], [50], [143] |
| | EGDMA + NIPAAm | TBPO | [62] |
| Ring opening | EO | BE3-DEE | [180] |
| | IBF | — | [181] |
| | MDO | TBPO | [37] |
| | Trioxane | — | [182] |

^{a)}Only iCVD chemistires, that is, excludes piCVD, iPECVD, oCVD, and other polymer CVD variants.
^{b)}4AS = 4-amino styrene; 4VP = 4-vinyl pyridine; AA = acrylic acid; CHMA; D₃; D₄; DEAAm = diethyl acrylamide; DEAEA = diethylaminoethyl acrylate; DEGDVE = di(ethylene glycol) divinylether; DFHA = dodecafluoroheptyl acrylate; DMAAm = dimethyl acrylamide; DMAEMA = N,N-dimethlaminoethyl methacrylate; DMAMA = dimethlaminomethyl styrene; DPAEMA = 2-diisopropylaminoethyl methacrylate; DVB = divinyl bensen; EA; EGDA; EGDMA = ethylene glycol dimethacrylate; EO = ethylene oxide; FMA = furfuryl methacrylate; GMA; HEMA; HFDMA = heptadecafluorodecyl methacrylate; HFPO; HVDS; IBF = isobensofuran; mDEB = m-diethynyl bensene; MAA = methacrylic acid; MAH = maleic anhydride; MCA = methyl α-chloroacrylate; MDO = 2-methylene-1,3-dioxepane; MMA = methyl methacrylate; nBA; nBMA = n-butyl methacrylate; nHA = n-hexyl acrylate; nPA = n-pentyl acrylate, nPrA = n-propyl acrylate; newPMA = neopentyl methacrylate; NIPAAm = N-isopropyl acrylamide; NVCL = N-vinyl caprolactam; oNBMA = o-nitrobenyl methacrylate; PFDA; PFEMA = perfluoroalkylethyl methacrylate; PFM = pentafluorophenyl methacrylate; PhAc = phenyl acetylene; PrgA = propargyl acrylate; PrgMA = propargyl methacrylate; S= styrene, tBA = t-butyl acrylate; V₃D₃; V₄D₄ = tetravinyltetramethyylcyclotetrasiloxane.
^{c)}BF₃-DEE = boron trifluoride-diethyl etherate; PFBSF = perfluorobutanesulfonyl fluoride; PFOSF; TAPA; TBPO; TBPOB.
^{d)}References exclude reviews.
^{e)}References with comonomers are duplicated depending on which monomer in the comonomer series is cited first.
^{f)}References in bold contain kinetic data.

In embodiments of the invention, said “initiating polymerization” may be by any appropriate initiation method. For example, in some embodiments, polymerization is initiated via photoinitiation (e.g., with light). In particular embodiments, polymerization is initiated by introducing polymerization initiator into the reactor.

In some embodiments, the polymerization initiator generates a radical that initiates a free radical polymerization upon contacting condensed droplets.

In some embodiments, the radical is generated by contacting the polymerization initiator with a heated filament array.

In some embodiments, the inventive method comprises introducing an additional agent (e.g., a therapeutic agent, for example, an anti-cancer agent, such as chlormethine) into the reactor.

In some embodiments, an additional agent (for example, a therapeutic agent, e.g., an active pharmaceutical ingredient (API), such as an anti-cancer agent or chemotherapeutic agent) is incorporated into polymer particles (e.g., nanoparticles) by delivering reagents in the vapor phase and avoiding the use of solvents. A substrate is placed into a vacuum chamber on a cooled stage. A therapeutic molecule or therapeutic molecules and/or a monomer or monomers are vaporized, delivered to the chamber, and condensed or solidified and/or polymerized on the substrate. In some embodiments, an outer shell is formed by vaporizing monomer(s) and delivering them to the chamber, and condensing and/or polymerizing on the substrate. In some embodiments, the polymerization is initiated by a vapor phase initiator (other initiating techniques include, e.g., photoinitiation), which polymerizes the droplet yielding a polymer particle loaded with the therapeutic agent with potentially programmable pharmacokinetics.

In some embodiments, the method comprises:
a) introducing an additional agent into the reactor;
b) introducing vapor-phase reagents into a reactor having a substrate;
c) forming condensed droplets of the reagents on the substrate;
d) introducing polymerization initiator into the reactor; and
e) polymerizing the condensed droplets of the reagents.

In some embodiments, a) is performed before b). In some embodiments, b) is performed before a). In some embodiments, a) and b) are performed at the same time. In various embodiments, c) is performed after b). In some embodiments, c) is performed after a). In some embodiments, c) is performed before a).

Vapors of some molecules can undergo two types of phase changes at a cooled surface. (1) If the vapor transitions into a liquid at the surface, it is called condensation and this forms either droplets (dropwise condensation) or films (filmwise condensation) of liquid. (2) Alternatively, a vapor can turn directly to a solid at a surface, and this is called deposition (there is no liquid phase involved). This is the case, for example, in certain embodiments utilizing chlormethine; it deposits on the substrate surface as a solid. The vapor to solid transition, called deposition, is the opposite of sublimation. The deposition phase change produces a thin layer of the reagent as a solid at the surface. Then, when a monomer condenses afterwards, the solid layer is dissolved into the liquid monomer.

In some embodiments, both the additional agent and the reagents are introduced into the reactor and both are condensed. However, in particular embodiments, one of either the additional agent or the reagents is introduced and, e.g., condensed or deposited, then the other is introduced and condensed or deposited, sequentially.

In some embodiments, the additional agent is condensed onto the substrate. In other embodiments, the additional agent is deposited onto the substrate (deposited meaning going from vapor to solid rather than vapor to liquid).

In some embodiments, introducing an additional agent into the reactor comprises vaporizing the additional agent into the reactor.

In some embodiments, introducing an additional agent into the reactor comprises sublimating the additional agent into the reactor.

In some embodiments, after the additional agent and vapor-phase reagents are introduced into the reactor, the additional agent disperses or dissolves within the condensed droplet of the reagents.

In various embodiments, the additional agent is capable of vaporization or sublimation without degradation/pyrolysis (unless the degradation byproduct is of value) in order to introduce it to the reactor as a vapor. This is species dependent; for example, 5-fluorouracil sublimes at 190-200 C at 0.1 mm Hg, but decomposes at 283 C. In some embodiments, the one or more additional agent sublimes at 100 to 300° C. (e.g., 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300° C.), including any and all ranges and subranges therein.

In various embodiments, the additional agent is capable of condensation (vapor to liquid) or deposition (vapor to solid) at a cooled surface at the temperature/pressure conditions achievable in a reactor setup. In some embodiments, the stage is cooled using liquid nitrogen.

In various embodiments, the additional agent is soluble (if deposited at the substrate surface) or miscible (if condensed at the substrate surface) in order to be taken up by the condensing monomer droplets prior to polymerization. The monomer may be partially soluble, in which case it would be desirable to achieve at least 1 mol % of the solute species.

In some embodiments, the additional agent comprises:
a therapeutic agent (for example, an anti-cancer agent, such as a chemotherapeutic agent, e.g., chlormethine, 5-fluorouracil, camustine, ifosfamide, thiotepa, etc.);
an anti-cancer agent (for example, curcumin, kaempferol, paclitaxel, resveratrol, silamarin, vincristine, etc.);
an antimicrobial agent (for example, artemisinim, caffeic acid, capsaicin, coumarin, eugenol, menthol, etc.);
an anti-inflammatory agent (for example, capsaicin, colchicine, curcumin, epigallocatechin-3-gallate, quercetin, resveratrol, etc.);
a neuroprotective agent (for example, bacoside A, bilobalide, curcumin, galantamine, ginsenosides, withaferin A);
an antioxidant agent (for example, curcumin, cyanidin, gingerol, Ginkgo biloba, glycyrrhizin, quercetin, etc.);
a cardiovascular protection agent (for example, berberine, curcumin, dihydrotanshinone, quercetin, resveratrol, etc.);
essential oils, metals (silver ions, copper ions), zinc oxide, graphene oxide or carbon nanotubes, photoactive compounds (titanium dioxide, benzophenone, MoS₂, MnO₂, zinc oxide, gold nanoparticles), eugenol, menthol, eucalyptol, capsaicin, polyphenols, etc.;
a fuel agent, for example, cerium oxide+H₂O₂, calcium carbonate+acid, catalysts (e.g., aluminum oxide, copper oxide, silver oside, iron oxide, cobalt oxide), etc.; or
a diagnostic and/or imaging agent, for example, fluorescent molecules, iodine, barium, supramagnetic iron oxide, bismuth, gold, an agent from Table I below, etc.,
or a combination thereof.

TABLE 1
Imaging/Contrast Agents

| Imagine | | | Sensitivity to | Nanoparticulate |
|---|---|---|---|---|
| method | Advantages | Disadvantages | contrast agents | contrast agents |
| MRI | High spatial resolution | Low sensitivity to | Milli- to | Gadolinium-containing probes¹⁹ |
| | (~10-500 μm) | contrast agents | micromolar | (Ultrasmall) superparamagnetic |
| | Unlimited penetration depth | High costs | | iron oxide |
| | Excellent soft tissue contrast | Time-intensive | | nonoparticles [(U)SPIO]²⁰ |
| | Versatile options for structural, | | | Paramagnetic liposomes and |
| | functional and metabolic | | | polymers²¹⁻²² |
| | tissue characterization | | | ParaCEST agents²² |
| | | | | Hyperpolarized probes²⁴ |
| CT | High resolution (~20-200 μm) | Insufficient soft tissue | Millimolar | Iodine-based micelles and |
| | Unlimited penetration depth | contrast without | | liposomes^{25,26} |
| | Good soft tissue contrast after | injection of contrast agents | | Barium-based nanoparticles²⁷ |
| | inection of contrast agent | Radiation exposure | | Gold-based nonapoarticles^{28,29} |
| | Low costs | Low sensitivity to | | Bismutin nanoparticles³⁰ |
| | Fast | contrast agents | | |
| Ultrasound | High temporal and spatial | User dependence | Single MB | Targeted and non-targeted |
| | resolution (~50-100 μm) | Not approprate for whole | detectable | gas-filled microbubbles³¹⁻³³ |
| | Rapidly aperable | body imaging | | Nanobubbles³⁴ |
| | Real-time imaging | | | Air-releasing polymers³⁵ |
| | Low costs | | | |
| Optical imaging | High sensitivity for | Low penetration depth | Nanomolar | Near-infrared fluorochrome- |
| | contrast agents | (<10 cm) | | labelled nanoparticles³⁶ |
| | Broad range of probes | High background signal | | Quantum dots³⁷ |
| | Low costs | Sensitive to artefacts | | Fluorescent nanoparticle probes³⁸ |
| Photoacoustic | High sensitivity | Limited penetration depth | Milli- to | Gold nanoparticles, gold nanorods³⁹ |
| imaging | Real-time imaging | (up to ~5-6 cm) | nanomolar | Carbon nanotubes⁴⁰ |
| | Low costs | Relatively low specificity | | Fluorescent/dye-loaded |
| | | to contrast agents | | nanoparticles⁴¹ |
| | | (signal from haemoglobin) | | |
| Positron emission | Very high sensitivity | Low spatial resolution (1-2 mm) | Picomolar | Radioactive contrast agents |
| tomography | Deep penetration depth | No anatomical information | | (e.g. radiolabeled |
| | Quantitative | Radiation exposure | | gold nanoshells)⁴² |
| | | High costs | | Polymeric nanoparticles⁴³ |
| Single photon | Very high sensitivity | Low spatial resolution (1-2 mm) | Picomolar | Technetium-labelled gold |
| emission CT | Unlimited penetration depth | No anatomical information | | nanoparticles⁴⁴ |
| | Long-circulating radionuclides | Radioactive probes | | Indium-labelled liposomes⁴⁵ |
| | | High cost | | Nano- and microcolloids⁴⁶ |

In some embodiments of the inventive method, the polymerizing step solidifies polymerized condensed droplets in place on the substrate as solid polymer particles.

In some embodiments, the polymer particles are hemispherical in shape (dome shaped).

In embodiments of the invention that use a polymerization initiator, any art acceptable polymerization initiator may be used. In some embodiments, the polymerization initiator is tert-butyl peroxide vapor that contacts a heated filament array to generate tert-butoxyl radicals that initiate free radical polymerization.

In some embodiments of the inventive method, during said introducing vapor-phase reagents into a reactor, the substrate is cooled. In some embodiments, the substrate is at a temperature of −10 to 80° C. (e.g., −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.), including any and all ranges and subranges therein.

With gaseous monomers present, condensation of the monomer onto the substrate surface is forced by increasing the chamber pressure and/or decreasing the temperature of the substrate to reach the saturation pressure of the monomer. Condensed droplets form at the saturation pressure with a hemispherical shape due to the high contact angle that results from the low interfacial energy. When the droplets are determined to be the desired size, a polymerization initiator is introduced/activated to solidify the condensed droplets in place.

In some embodiments of the inventive method, said introducing vapor-phase reagents into a reactor comprises introducing the reagents into an evacuated and/or isolated chamber of the reactor that houses the substrate.

In some embodiments, the substrate is functionalized (e.g., has a functionalized coating thereon). Persons skilled in the art are able to identify acceptable functionalizations or coatings, and it is contemplated that all such embodiments may be used in the invention. In some embodiments, the substrate comprises a perfluorinated polymer or poly(divinyl benzene).

In some embodiments, the substrate comprises an omniphobic coating that enables dropwise condensation on the substrate.

In some embodiments, the coating on the substrate is hydrophilic or hydrophobic or omniphobic.

In some embodiments, the coating on the substrate has a thickness of 50 to 500 nm (e.g., 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm), including any and all ranges and subranges therein (e.g., 100-200 nm).

In some embodiments of the inventive method, following the polymerizing, the polymer particles are dry due to the solvent free nature of the method. In some embodiments, the inventive method utilizes less than 20 wt % (e.g., less than 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, or 0.1 wt %) solvent based on the total weight of reactants (e.g., vapor reactants) added to the reactor.

In some embodiments of the inventive method, following the polymerizing, the polymer particles have not been exposed to liquid and/or solvent during the method. In some embodiments, the only exposure to a liquid is condensed liquid from vaporized reagents and/or additional agent on the substrate.

In some embodiments, following the polymerizing, atoms from any coating present on the substrate are not present in the polymer particle (in other words, no part of the coating becomes part of the polymer particle).

In some embodiments, the polymerizing is complete within less than 200 seconds (for example, in some embodiments, the polymerizing is complete within less than 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 seconds).

In some embodiments, the polymer particles have a size of 0.01 nm to 1,000,000 nm (e.g., 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 200000, 300000, 400000, 500000, 600000, 700000, 800000, 900000, or 1000000 nm), including any and all ranges and subranges therein.

In some embodiments, the inventive method is performed without dissolving or dispersing monomer(s) or reagents in solution.

In some embodiments, the inventive method introduces only monomers and initiator and optionally an additional agent (e.g., a therapeutic agent) to a solution-free system to prepare the polymer particles, thereby evading the requirement of purification steps and restrictions on access to monomers based on solubility.

In some embodiments, the inventive method does not utilize plasma.

In some embodiments, plasma is not generated during the inventive process.

In some embodiments, the inventive method does not utilize Plasma-Enhanced Chemical Vapor Deposition (PECVD).

In some embodiments, the inventive method is such that the polymer material of the polymer particle has a lower hydrogen content than a corresponding polymer material produced from PECVD would have due to the utilization of plasma in the PECVD deposition process.

In a second aspect, the invention provides a polymer particle comprising:
polymer material; and
optionally, mixed with the polymer material, an additional agent (e.g., a therapeutic agent).

Embodiments of the inventive polymer particle of the second aspect of the invention are prepared according to the first aspect of the invention.

In some embodiments, the additional agent is homogeneously or heterogeneously dispersed within, or mixed within, the polymer material.

In some embodiments, the polymer particle has a substantially uniform composition distribution (i.e., has a composition distribution that is at least 90%, e.g., at least 90, 91, 92, 93, 94, 95, 96, 97, 98, or 99% uniform).

In some embodiments, the polymer material is present, alone or mixed (e.g., substantially homogenously mixed) with other materials (e.g., a therapeutic agent), throughout the entire particle. Such embodiments exclude, for example, particles having a core that does not comprise polymer material.

In some embodiments, the polymer material in the polymer particle is present at a concentration or percentage close to the surface of the polymer particle (e.g., within the outermost 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 vol % of the particle) that is higher than, lower than, or substantially equal (e.g., within 1, 2, 3, 4, 5, 6, 7, 8, 9, or 10% variation) to the concentration or percentage of the polymer material in the middle of or inside the formed particle (e.g., at the centermost/innermost 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 vol % of the particle).

In some embodiments, the polymer particle comprises a therapeutic agent and the concentration or percentage of the therapeutic agent close to the surface of the polymer particle (e.g., within the outermost 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 vol % of the particle) is higher than, lower than, or substantially equal (e.g., within 1, 2, 3, 4, 5, 6, 7, 8, 9, or 10% variation) to the concentration or percentage of the drug in the middle of or inside the formed particle (e.g., at the centermost/innermost 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 vol % of the particle).

In some embodiments, the therapeutic agent is distributed throughout the entire polymer particle.

In some embodiments, the polymer particle comprises 1 to 100 wt % polymer material (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 wt %), including any and all ranges and subranges therein (e.g., 40-100 wt %, 60-100 wt %, 80-100 wt %, etc.).

In some embodiments, the polymer particle comprises 0 to 99 wt % therapeutic agent (e.g., 0, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt %), including any and all ranges and subranges therein (0.001 to 50 wt %, 0.001 to 40 wt %, 0.001 to 30 wt %, 0.001 to 20 wt %, 0.001 to 10 wt %, etc.)

In some embodiments of the polymer particle, the polymer material contains a predominant chain, and end groups of the predominant chain are a methyl group on one end and a monomer on another end, e.g., a monomer end group of formula:

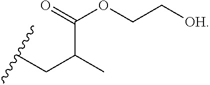 [SMILES: *CC(C)C(=O)OCCO]

In some embodiments, the predominant chain does not comprise tertbutoxyl end groups.

In some embodiments (e.g., when analyzed using matrix-assisted laser desorption ionization time-of-flight (MALDI-TOF) mass spectrometry), the polymer particle:
has a predominant chain having a methyl end group; and/or
has a predominant chain having a monomer group end group; and/or
has a predominant chain that does not have a tertbutoxyl end group;

and/or
has a predominant chain that does not have a tertbutoxyl end group.

In some embodiments, the particle is prepared via CDP, and:
the polymer material has a molecular weight higher than a corresponding polymer material prepared via iCVD (e.g., has a number averaged molecular weight (Mn) or a weight averaged molecular weight (Mw) greater than the corresponding polymer material prepared via iCVD; or
the polymer materials has longer polymer chains than a corresponding polymer material prepared via iCVD; or
the polymer materials has a lower polydispersity (PD) than a corresponding polymer material prepared via iCVD.

In some embodiments, the polymer material has a molecular weight Mw1, which is higher than a corresponding polymer material prepared via iCVD, having a molecular weight Mw2, wherein (Mw1−Mw2)/Mw2×100%≥5% (e.g., is ≥5, 6, 7, 8, 9, 10, 11, 12, 13, 14, 15, 16, 17, or 18%).

In some embodiments, the polymer material has a molecular weight Mn1, which is higher than a corresponding polymer material prepared via iCVD, having a molecular weight Mn2, wherein (Mn1−Mn2)/Mn2×100%≥5% (e.g., is ≥5, 6, 7, 8, 9, 10, 11, 12, 13, 14, 15, 16, 17, or 18%).

In some embodiments, the particle does not comprise:
a distinct outer coating containing the polymer material, the outer coating encapsulating an inner discrete particle; or
any component that was formed via CDP; or
residual solvent (e.g., from a polymerization); or
poly-para-xylylenes.

In some embodiments, the polymer particle comprises less than 10 wt % solvent (e.g., less than 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01 wt %) based on the total weight of the polymer particle.

In some embodiments, the entire polymer particle originates from one or more (e.g., 2, 3, 4, etc.) vapors. Such embodiments exclude, for example, pre-existing particles that could be disposed in a reactor chamber, and coated with polymer material.

In some embodiments, the polymer particle is not spherical.

In some embodiments, the particle is in the shape of a hemisphere.

In some embodiments, the polymer particle is not porous.

In some embodiments, the polymer particle is porous.

In some embodiments, the polymer particle is non-porous or has pore structures present having a size of less than 10 nm (e.g., an average pore size of less than 10, 9, 8, 7, 6, 5, 4, 3, 2, or 1 nm). In other embodiments, the polymer particles have larger pore sizes.

In some embodiments, the polymer particle is a solid, template-free polymer particle prepared without involving a liquid substrate.

In some embodiments, the polymer particles of the present invention and methods of making the same find use in:
Drug delivery
Injectable implants
Virus detection
Toxins/toxic metals detection
Soft robotics
Materials enhancements (e.g., durability enhancement, anti-corrosion)
Bioactive food compounds
Release of protective or nutritional elements in plants/agriculture
Environmental remediation
Nanolenses for enhanced microscopy
Adhesives
Antireflective surfaces
Fuel efficiency

In some non-limiting embodiments, the invention is as described in any one of the following clauses, wherein it is contemplated that any clause may be combined with another clause, unless clauses clearly conflict:

### Clauses

Clause 1. A method of synthesizing polymer particles, said method comprising:
introducing vapor-phase reagents into a reactor having a substrate;
forming condensed droplets of the reagents on the substrate;
initiating polymerization (e.g., by introducing polymerization initiator into the reactor or via photoinitiation); and
polymerizing the condensed droplets of the reagents, thereby forming polymer particles.

Clause 2. The method according to Clause 1, wherein the vapor-phase reagents include one or more monomers.

Clause 3. The method according to Clause 2, wherein the monomers comprise 2-hydroxyethyl methacrylate, divinylbenzene, 4-vinylpyridine, benzyl methacrylate, ethylene glycol dimethacrylate, 1-vinylimidazole, cyclohexyl methacrylate, 2-(dimethylamino)ethyl methacrylate, or glycidyl methacrylate, or any combination thereof.

Clause 4. The method according to any one of Clauses 1 to 3, wherein the polymerization initiator generates a radical that initiates a free radical polymerization upon contacting the condensed droplets.

Clause 5. The method according to Clause 4, wherein the radical is generated by contacting the polymerization initiator with a heated filament array.

Clause 6. The method according to any one of the preceding Clauses, further comprising:
introducing an additional agent (e.g., a therapeutic agent, for example, an anti-cancer agent, such as chlormethine) into the reactor.

Clause 7. The method according to Clause 6, wherein the method is performed in the following order:
introducing an additional agent into the reactor;
introducing vapor-phase reagents into a reactor having a substrate;
forming condensed droplets of the reagents on the substrate;
introducing polymerization initiator into the reactor; and
polymerizing the condensed droplets of the reagents.

Clause 8 The method according to Clause 6 or 7, wherein said introducing an additional agent into the reactor comprises sublimating the additional agent into the reactor.

Clause 9. The method according to any one of Clauses 6 to 8, wherein after the additional agent and vapor-phase reagents are introduced into the reactor, the additional agent disperses or dissolves within the condensed droplet of the reagents.

Clause 10. The method according to any one of Clauses 6 to 9, wherein the additional agent comprises:
a therapeutic agent (for example, an anti-cancer agent, such as a chemotherapeutic agent, e.g., chlormethine, 5-fluorouracil, camustine, ifosfamide, thiotepa, etc.);
an anti-cancer agent (for example, curcumin, kaempferol, paclitaxel, resveratrol, silamarin, vincristine, etc.);
an antimicrobial agent (for example, artemisinim, caffeic acid, capsaicin, coumarin, eugenol, menthol, etc.);
an anti-inflammatory agent (for example, capsaicin, colchicine, curcumin, epigallocatechin-3-gallate, quercetin, resveratrol, etc.);
a neuroprotective agent (for example, bacoside A, bilobalide, curcumin, galantamine, ginsenosides, withaferin A);
an antioxidant agent (for example, curcumin, cyanidin, gingerol, Ginkgo biloba, glycyrrhizin, quercetin, etc.);
a cardiovascular protection agent (for example, berberine, curcumin, dihydrotanshinone, quercetin, resveratrol, etc.);
essential oils, metals (silver ions, copper ions), zinc oxide, graphene oxide or carbon nanotubes, photoactive compounds (titanium dioxide, benzophenone, MoS₂, MnO₂, zinc oxide, gold nanoparticles), eugenol, menthol, eucalyptol, capsaicin, polyphenols, etc.;
a fuel agent, for example, cerium oxide+H₂O₂, calcium carbonate+acid, catalysts (e.g., aluminum oxide, copper oxide, silver oxide, iron oxide, cobalt oxide), etc.; or
a diagnostic and/or imaging agent, for example, fluorescent molecules, iodine, barium, supramagnetic iron oxide, bismuth, gold, etc.,
or a combination thereof.

Clause 11. The method according to any one of the preceding Clauses, wherein said polymerizing solidifies polymerized condensed droplets in place on the substrate as solid polymer particles.

Clause 12. The method according to any one of the preceding Clauses, wherein the polymer particles are hemispherical in shape.

Clause 13. The method according to any one of the preceding Clauses, wherein the polymerization initiator is tert-butyl peroxide vapor that contacts a heated filament array to generate tert-butoxyl radicals that initiate free radical polymerization.

Clause 14. The method according to any one of the preceding Clauses, wherein, during said introducing vapor-phase reagents into a reactor, the substrate is cooled.

Clause 15. The method according to any one of the preceding Clauses, wherein said introducing vapor-phase reagents into a reactor comprises introducing the reagents into an evacuated, isolated chamber of the reactor, said chamber housing the substrate.

Clause 16. The method according to any one of the preceding Clauses, wherein the substrate is functionalized (e.g., has a functionalized coating thereon).

Clause 17. The method according to Clause 16 wherein the substrate comprises a perfluorinated polymer or poly(divinyl benzene).

Clause 18. The method according to Clause 16 or Clause 17, wherein the substrate comprises an omniphobic coating that enables dropwise condensation on the substrate.

Clause 19. The method according to any one of the preceding Clauses, wherein following said polymerizing, the polymer particles are dry due to the solvent free nature of the method.

Clause 20. The method according to any one of the preceding Clauses, wherein following said polymerizing, the polymer particles have not been exposed to liquid or solvent during the method.

Clause 21. The method according to any one of the preceding Clauses, wherein following said polymerizing, atoms from any coating present on the substrate are not present in the polymer particle.

Clause 22. The method according to any one of the preceding Clauses, wherein said polymerizing is complete within less than 120 seconds (e.g., less than 60 seconds).

Clause 23. The method according to any one of the preceding Clauses, wherein the polymer particles have a size of 0.01 nm to 1,000,000 nm.

Clause 24. A polymer particle prepared according to the method of any one of the preceding Clauses.

Clause 25. A polymer particle comprising:
polymer material; and
optionally, mixed with the polymer material, an additional agent (e.g., a therapeutic agent).

Clause 26. The polymer particle according to Clause 25, wherein the therapeutic agent is homogeneously or heterogeneously dispersed within polymer material.

Clause 27. The polymer particle according to any one of Clauses 24-26, wherein the polymer material contains a predominant chain, and end groups of the predominant chain are a methyl group on one end and a monomer on another end.

Clause 28. The polymer particle according to Clause 27, wherein the monomer end groups are of formula:

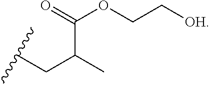 [SMILES: *CC(C)C(=O)OCCO]

Clause 29. The polymer particle according to any one of Clauses 24-28, wherein the predominant chain does not comprise tertbutoxyl end groups.

Clause 30. The polymer particle according to any one of Clauses 24 to 29, wherein the particle is prepared via CDP, and wherein:
the polymer material has a molecular weight higher than a corresponding polymer material prepared via iCVD (e.g., has a number averaged molecular weight (Mn) or a weight averaged molecular weight (Mw) greater than the corresponding polymer material prepared via iCVD; or
the polymer materials has longer polymer chains than a corresponding polymer material prepared via iCVD; or
the polymer materials has a lower polydispersity (PD) than a corresponding polymer material prepared via iCVD.

Clause 31. The polymer particle according to Clause 30, wherein the polymer material is PHEMA.

Clause 32. The polymer particle according to any one of Clauses 24 to 31, wherein the particle does not comprise: a distinct outer coating containing the polymer material, the outer coating encapsulating an inner discrete particle.

Clause 33. The polymer particle according to any one of Clauses 24 to 32, wherein the particle is in the shape of a hemisphere.

### EXAMPLES

The invention will now be illustrated, but not limited, by reference to the specific embodiments described in the following examples.

### Example A—Preparation of Embodiments of Particles Synthesized Via CDP

Surface Layer Application to Substrate: All purchased chemicals were used as received without modification. Silicon (Si) wafers (Pure Wafer) coated with fluorinated thin films were prepared using the initiated Chemical Vapor Deposition (iCVD) technique in a custom built reactor corresponding to that depicted in FIG. 2 comprised of parts and dimensions detailed in T. B. Donadt, R. Yang, Adv. Mater. Interfaces 2021, 5, 2001791.

A silicon wafer was placed in an iCVD reactor chamber held at 400 mTorr on a temperature-controlled stage maintained at 35° C. A glass jar containing 1H, 1H,2H,2H-perfluorofecyl acrylate (PFDA, Sigma-Aldrich, 97%) was heated to 80° C. and the monomer vapors were delivered to the vacuum chamber through a needle valve at flow rate of 0.15 sccm. Argon carrier gas and tert-butyl peroxide (TBPO) were also delivered into the chamber through mass flow controllers at 2.00 sccm and 0.60 sccm respectively. A filament array composed of 0.5 mm copper/nickel wire (55% Cu/45% Ni, Goodfellow) was positioned 3 cm above the substrate stage and heated to 300° C. to thermally decompose TPBO into tert-butoxyl and methyl radicals. Contact of radicals with PFDA molecules adsorbed to the Si wafer initiated the thin film polymerization. The deposition thickness was observed in real time using an interferometer with a 633 nm helium-neon laser (JDS Uniphase) until a coating between 100 and 200 nm was formed (approximated due to significant surface roughness).

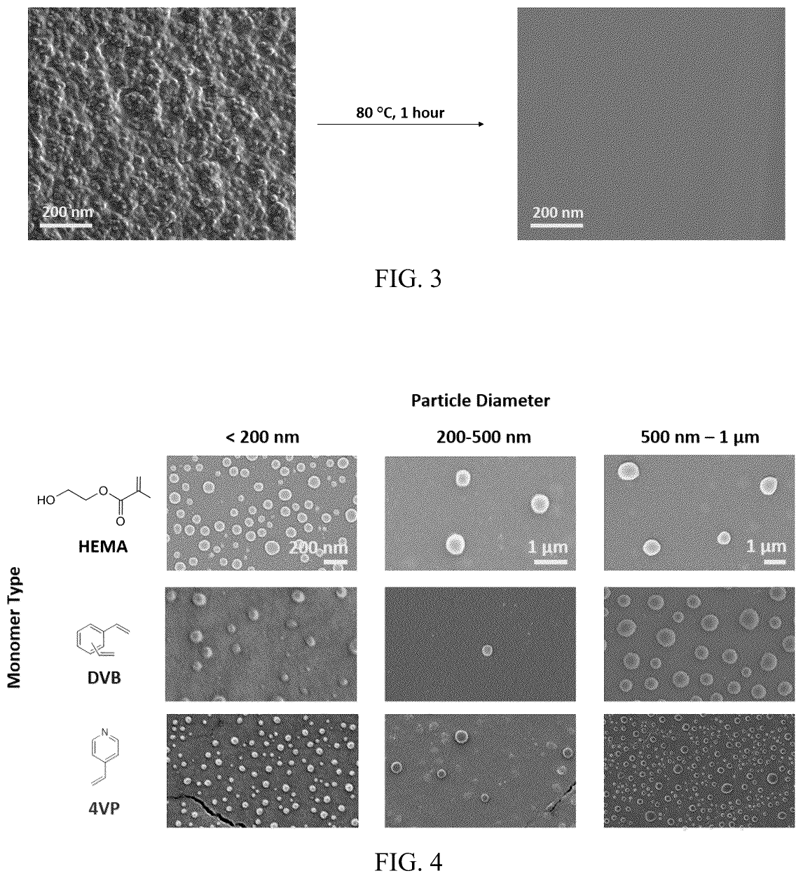

Fluorinated polymer thin films of poly(1H,1H,2H,2H-perfluorodecyl acrylate) (“poly (PFDA)” or “PPFDA”) thin films can exhibit crystalline domains that yield rough surfaces (see FIG. 3, which depicts flattening of a fluorinated base layer). To ensure a flat layer for the condensed droplet polymerization, PPFDA films were placed in an oven set to 80° C. for one hour. At this temperature, roughness on the order of hundreds of nanometers is reduced to picometer range by eliminating organized crystalline domains responsible for surface protrusions.

Nanoparticle synthesis via condensed droplet polymerization (CDP): Silicon wafer substrates with fluorinated surface layers were placed into the iCVD reactor atop a thermoelectric cooling device (TEC, VT-127-1.0-1.3-71, TE Technology). The thermoelectric cooling module enables fine-tuned control of substrate temperatures to direct particle growth. A ceramic thermal compound (Céramique™ 2, Arctic Silver) was used to secure the TEC to underlying stage that was held at 20° C. stage and the reactor chamber was evacuated to below 5 mTorr. The TEC was cooled to below 15° C. by the application of electrical current from a DC power source (1715A, B&K Precision) and the filament array was heated to approximately 300° C. using another DC power source of the same type. A throttle valve (253B, MKS Instruments) was then closed at the outlet to the vacuum pump to isolate the reactor chamber containing the substrate. Monomer stock was heated in a glass jar to generate vapors that were metered into the reactor through a needle valve until the saturation pressure was reached [2-hydroxyethyl methacrylate (HEMA (Sigma-Adlrich, >99%) was heated to 80° C., 4-vinylpyridine (4VP (Sigma-Aldrich, 95%)) was heated to 50° C., and divinylbenzene (DVB (Sigma-Aldrich, 80%)) was heated to 65° C.]. Saturation pressure was dependent on the substrate temperature and type of monomer and condensation occurred between 10-150 mTorr. Droplet formation was monitored with two devices: the aforementioned laser interferometer that dropped precipitously upon droplet formation and a digital microscope (VHX 970F, Keyence) that showed the droplets as they formed. Once proper droplet size was achieved, monomer flow was stopped. TBPO was delivered to the chamber at 1.80 sccm for 15-30 seconds to initiate polymerization. After TBPO flow stopped, the contents of the chamber were allowed to continue polymerizing at a stable pressure for an additional 15 seconds. Finally, the throttle valve at the outlet to the vacuum pump was opened to stop the reaction and clear the chamber of all vapors and unreacted monomer.

Confirmation of successful synthesis of PNP's: To confirm the successful synthesis of PNPs, the substrates were observed as described below using scanning electron microscopy (SEM). PNPs comprised of poly(4VP) (P4VP), poly (DVB) (PDVB), and poly (HEMA) (PHEMA) were synthesized atop the PPFDA-coated substrates following the same procedure. These PNPs represent varying degrees of hydrophilicity/phobicity, functionalizability, hydrogel-forming, and crosslinking, all synthesized using the same technique. FIG. 6 is an SEM image of an embodiment of inventive PHEMA nanoparticles. The SEM image illustrates the circular profile from a top-down view, but the result of the CDP process is hemispherical nanoparticles in the shape of a sphere segment with a flat side where the droplet contacted the underlying substrate. By imaging the PNPs from a side angle, the hemispherical shape was revealed and a view of the bottom revealed complete polymerization to the core that is farthest from the liquid-vapor surface where initiator radicals contact the droplet.

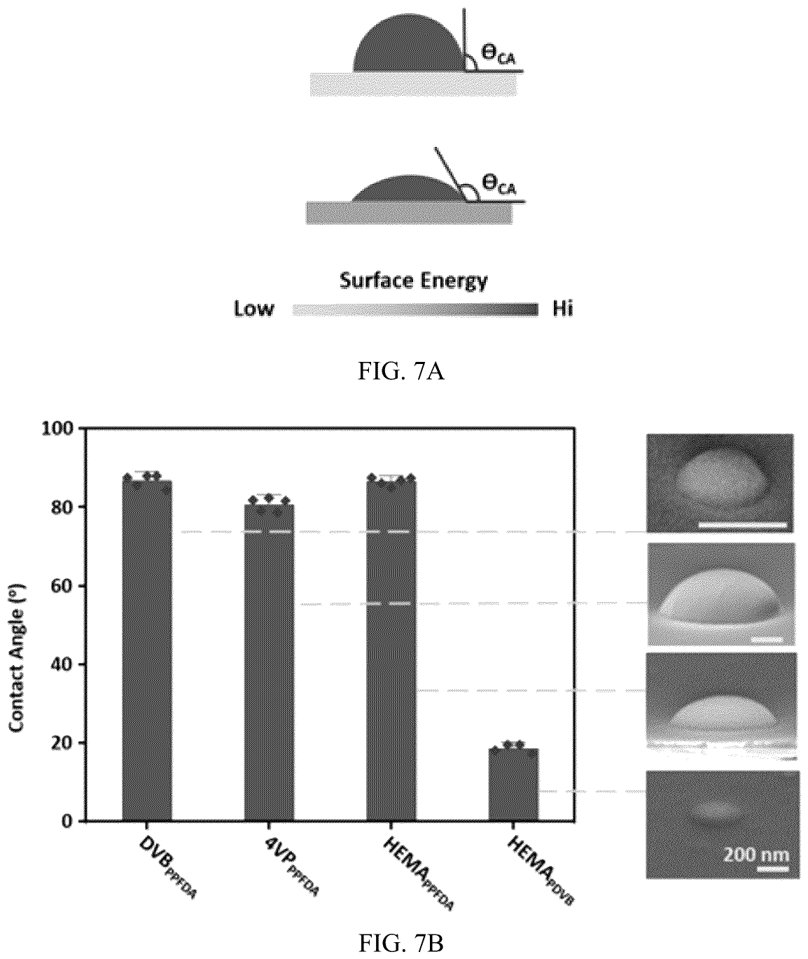

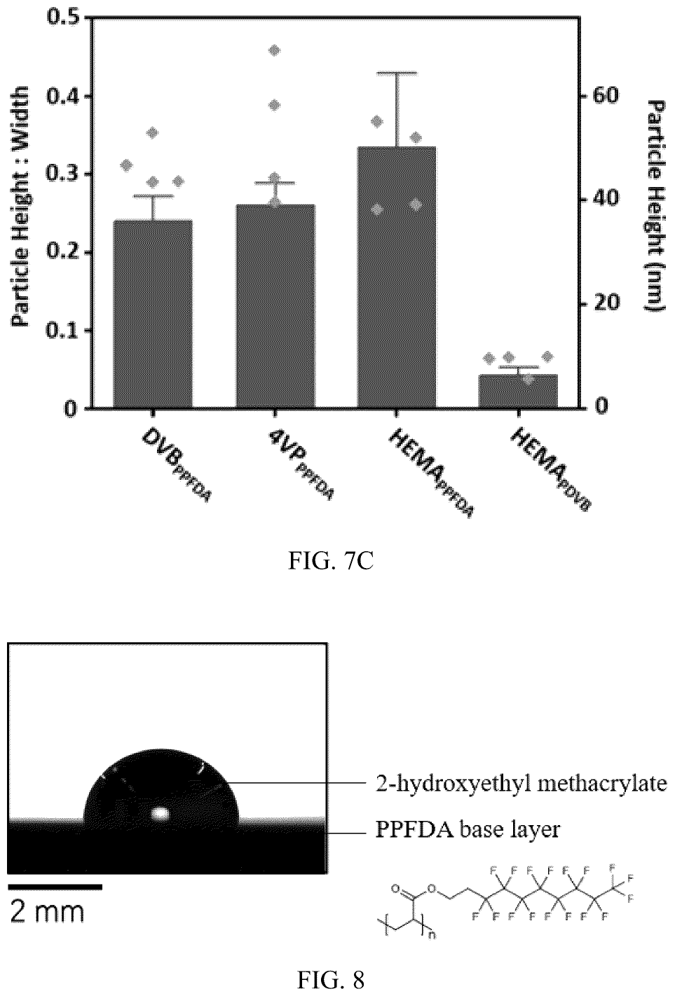

CDP represents a versatile platform for hemispherical PNP synthesis, as it accomplishes shape programmability without a nano-structured template using the surface energy relationships between the substrate surface chemistry and the liquid monomer. When considering a hydrophilic monomer, a substrate surface with a low surface energy will lead to less wetting by a condensed droplet and a higher contact angle at the edge (FIG. 7A). On the other hand, a substrate surface with a high surface energy will lead to more wetting and a lower contact angle. Controlling the contact angle controls the diameter to height ratio of the liquid droplet and the resulting solid polymer nanoparticle; thus, by choosing a substrate chemistry to produce a specific contact angle of the liquid monomer, the shape may be programmed.

Characterization of polymer nanoparticle chemistry: FTIR spectra of polymer films and nanoparticles on Si wafers were collected using a Bruker VERTEX Series V80v spectrometer in transmission mode and a mercury cadmium telluride (MCT) detector. Spectra recorded across a range of 4000-600 cm⁻¹ (4 cm⁻¹ resolution) were averaged over 128 scans, background corrected using a bare Si wafer, and baseline corrected. A P4VP thin film was prepared for FTIR analysis according the iCVD procedure in Surface Layer Application to Substrate with 4VP as a monomer and the following conditions: 4VP, TBPO, and argon flow rates of 3.7, 0.5, and 1.0 sccm, respectively; reactor chamber pressure of 400 mTorr; filament array temperature of approximately 250° C.; stage temperature of 25° C. A PDVB thin film was prepared for FTIR analysis according to the same procedure with DVB as a monomer and the following conditions: DVB, TBPO, and argon flow rates of 0.6, 0.5, and 0.8 sccm, respectively; reactor chamber pressure of 400 mTorr; filament array temperature of approximately 250° C.; stage temperature of 15° C. A PHEMA thin film was prepared according the same procedure with HEMA as a monomer and the following conditions: HEMA, TBPO, and argon flow rates of 0.5, 0.9, and 1.3 sccm, respectively; reactor chamber pressure of 300 mTorr; filament array temperature of approximately 270° C.; stage temperature of 30° C.

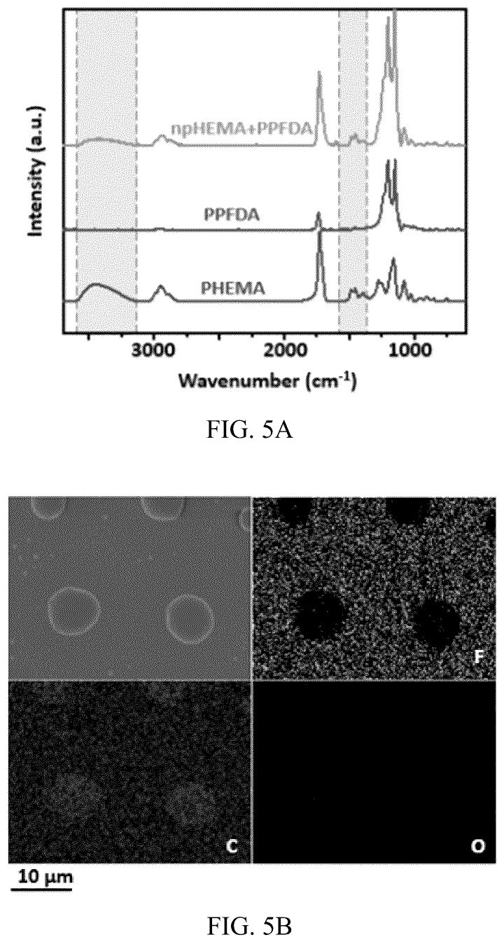

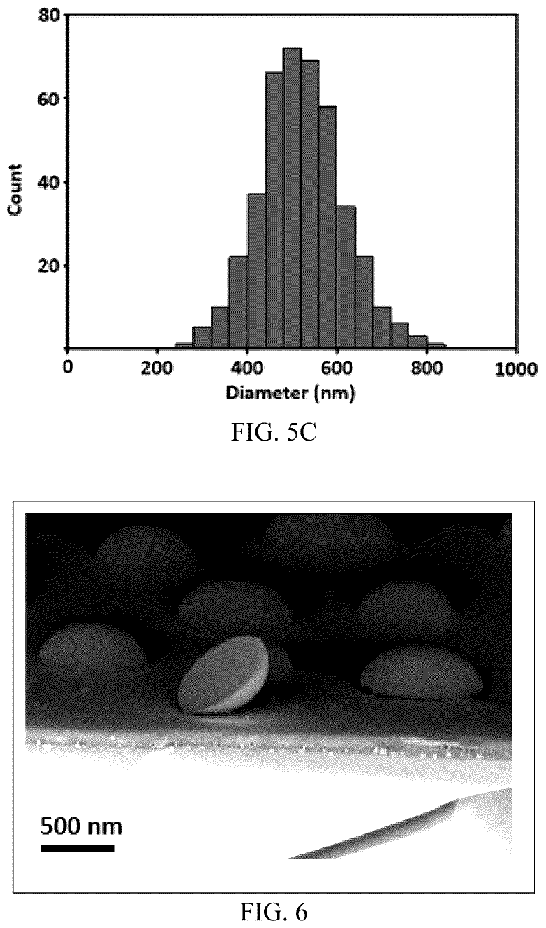

To confirm the chemical composition of the nanoparticles, the FTIR spectra of the PPFDA base layer, the base layer after being covered PHEMA nanoparticles after CDP, and a PHEMA polymer thin film (FIG. 5A) were compared. Peaks associated with the base layer and PHEMA thin film are preserved in the combined spectrum containing the nanoparticles on the base layer. Peaks from the PHEMA thin film that do not overlap with peaks from the PPFDA base layer and are visible in the combined spectrum confirm the chemical composition of the nanoparticles as PHEMA. One band highlights the broad peak above 3000 cm⁻¹ that identifies the O—H stretching vibration and another highlights the peak at 1457 cm⁻¹ associated with bending of C—H bond in the polymer backbone and the side chain's ethyl moiety. Absent from each spectrum is a peak from C═C bonds (1660-1610 cm⁻¹) that would signify unreacted monomers and confirms complete polymerization. Similar plots were obtained for the comparison of spectra containing PDVB and P4VP (not pictured).

SEM and EDX were performed on a Zeiss GeminiSEM 500 on samples that had been coated with approximately 3 nm of gold/palladium. Acceleration voltages used were 1 kV for SEM images and 3 kV for EDX element mapping. The penetration of the x-ray necessitated the synthesis of larger PHEMA particles with diameters around 10 μm in order to differentiate the chemistry of the particles from the base layer. A lack of fluorine atoms detected within the particles indicates that the particles are not a morphological feature of the PPFDA base layer, but are a new chemistry added on top. The higher concentration of carbon and oxygen atoms are indicative of the PHEMA chemistry in the particles captured in FIG. 5B.

To further elucidate components of the reaction mechanism in CDP that are unique from traditional iCVD thin film polymerization, PHEMA CDP nanoparticles and PHEMA iCVD thin films were analyzed using matrix-assisted laser desorption ionization time-of-flight (MALDI-TOF) mass spectrometry. MALDI-TOF analysis revealed an increase in polymer chain length as a result of CDP (Mn=2909.74 Da, Mw=3422.83 Da, polydispersity [PD]=1.20) compared to iCVD (Mn=2159.69 Da, Mw=2868.03 Da, PD=1.32). End group analysis revealed that the predominant polymer chain type resulting from iCVD featured a tert-butoxide group at one end and a methyl group at the other. Alternatively, the predominant polymer type resulting from CDP featured a tert-butoxide group at one end and a HEMA unit at the other end. This indicates that a chain transfer or disproportionation event is the most common termination event in CDP. Autoacceleration is known to generate hot spots that promote chain transfer events, hinting that features of the bulk polymerization influence the dominant chain termination type in CDP along with the droplet geometry in which propagating chains are more likely to meet unreacted monomers in the droplet compared to other propagating chains or impinging gaseous initiator molecules. Characterization here focuses on PHEMA to exemplify the successful synthesis of PNPs by CDP, though any of the chemistries may be analyzed by the same techniques. The biocompatibility of PHEMA makes the polymer a promising candidate for a variety of biomedical applications that may be advanced by Applicant's CDP technique.

For MALDI-TOF, 3 CDP syntheses of PHEMA particles were performed according to the above Nanoparticle synthesis via condensed droplet polymerization procedure. The polymer content (PHEMA particles and PPFDA base layer) was scraped off of each Si wafer substrate using a clean razor blade into a microcentrifuge tube into which methanol (100 μL) was added to dissolve only the PHEMA nanoparticles. The PHEMA thin film (approximately 3 mg) synthesized according to the protocol above was scraped off of the Si wafer substrate into a microcentrifuge tube into which methanol (100 μL) was added to dissolve the film. A stock matrix solution was prepared by dissolving α-cyano-4-hydroxycinnamic acid (20 μg, CHCA, Sigma-Aldrich, >98%) in methanol (1 mL). The polymer-containing solutions were mixed with the matrix solution and purified water in a ratio of 1:1:0.4 (polymer solution:matrix solution:water), vortexed, spotted onto the MALDI-TOF analysis plate, and allowed to dry completely. MALDI-TOF spectra were collected using a Bruker autoflex max in positive reflectron mode and analyzed using Polymerix (Sierra Analytics).

PHEMA nanoparticle diameter dispersity: SEM imaging was also used in conjunction with FIJI analysis to characterize the dispersity of nanoparticles diameters resulting from CDP of HEMA (FIG. 5C). Dispersity analysis was performed using FIJI. Nucleation that leads to condensation was expected to occur randomly across the surface. However, the resulting nanoparticles diameters were not randomly distributed across the entire nanoscale. Particles from the same synthesis were found be clustered around a mean with a modest dispersity in sizes. The histogram of FIG. 5C represents the analysis of 416 PHEMA nanoparticles from 10 SEM images of unique locations across the substrate. PHEMA nanoparticles from this synthesis were 519±92 nm in diameter. A Gaussian distribution with a coefficient of variation 0.18 was observed, a smaller value than may be expected due to the random nucleation events, which may be attributed to the diffusion length of the initiating radicals and flow conditions of the reactor.

Contact angle and aspect ratio analysis: Contact angle measurements were performed using a Ramé-Hart Model 500 contact angle goniometer. A pipette was used to dispense 5 μL of liquid HEMA, 4VP, and DVB onto the substrate for static contact angle measurements. Measurements were repeated 5 times for each monomer on the PPFDA substrate and 4 times for HEMA on the PDVB substrate.

The aspect ratio of nanoparticle height to diameter was measured using an Asylum Research MFP-3D-BIO atomic force microscope (AFM) in AC tapping mode. Scans were recorded across 1×1 μm and 0.5 Hz regions for each nanoparticle chemistry on the PPFDA substrate and 5×5 μm and 1 Hz for PHEMA on the PDVB substrate. Scans were repeated in 4 different areas across the substrate and particles were selected with diameters in the range of 120-265 nm. The profile of the selected particles, 4 of each kind, were traced across the center of the particle to determine the end-to-end distance and base-to-tip height.

Contact angle measurements were recorded for liquid droplets of HEMA, DVB, and 4VP on the PPFDA substrate surface. On the flattened PPFDA base layer, contact angles were 86.5±1.6° for DVB, 80.6±1.7° for 4VP, and 86.4±1.0° for HEMA (FIG. 7B). SEM images taken at a side angle confirm the near 90° contact angles and equivalent hemispherical profiles of the nanoparticles from each monomer on a PPFDA substrate. Importantly, this confirmed a reliable correlation between the contact angle of measurable macroscale droplets with the contact angle of nanoscale droplets which we did not observe until after polymerization. Despite a potential change in volume from liquid monomer to solid polymer, the contact angle observed at the macroscale persisted at the nanoscale upon formation of the polymerized product. Young's equation is often modified at the nanoscale where the line tension at the triple interface balances the surface tension, but this is generally observed to deviate from the macroscale at single-digit nanometer droplet radii which are smaller than the droplets analyzed here.

To raise the substrate surface energy, a substrate was coated with a PDVB thin film on which liquid HEMA exhibits at contact angle of 18.5±1.1°, significantly lower than the contact angle of HEMA on PPFDA. At the decreased contact angle on PDVB, the resulting PHEMA nanoparticle observed in SEM is flatter (e.g. has a lower height to diameter ratio). Atomic force microscopy (AFM) was used to trace the profile of the nanoparticles and quantify the height to diameter ratio (FIG. 7C). PNPs of PDVB and PHEMA on PPFDA exhibit higher aspect ratios of 0.24±0.021 and 0.26±0.019, respectively. The height to diameter ratio of PHEMA on PPFDA is also elevated (0.33±0.063), but is flattened on PDVB (0.043±0.0072). Accordingly, altering the surface chemistry and surface energy of the substrate surface can program the shape of the resulting nanoparticles without the use of laborious surface templating or implementation of nano-structures as in the case of hemispherical polymer nanoparticles derived from Janus particles. Once separated from the surface, the nanoparticles retain their rounder or flatter shape established by the contact angle of the monomer droplet prior to polymerization in CDP.

### Example B—Chain Length & End Group Testing of Additional CDP Polymer Particle Embodiments

Matrix-assisted laser desorption/ionization coupled to time-of-flight mass spectrometry (MALDI-TOF) was used to compare chain length and end groups of poly(2-hydroxyethyl methacrylate) (PHEMA) in particles made via CDP (the protocol discussed in Example A) versus a conventional CVD thin film technique called initiated chemical vapor deposition (iCVD). Results are as follows:

| | PHEMA | PHEMA |
|---|---|---|
| | (CDP) | (iCVD) |
| Mn | 2944.16 Da | 2165.61 Da |
| Mw | 3417.51 Da | 2859.59 Da |
| PD | 1.16 | 1.32 |

| | End Group 1 | End Group 2 |
|---|---|---|
| CDP | 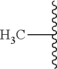 | 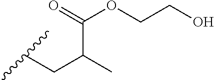 [SMILES: *CC(C)C(=O)OCCO] |
| iCVD | 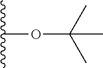 [SMILES: *OC(C)(C)C] | 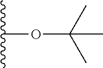 [SMILES: *OC(C)(C)C] |

Polymer chains from CDP were larger on average with a lower polydispersity (PD) compared to iCVD. The end groups of the predominant chain type in iCVD were tertbutoxyl groups on both ends. In CDP, the end groups of the predominant chain were methyl groups on one end (due to β-scission of the initiator at a warmer array temperature) and a monomer at the other, indicating the presence of termination by chain transfer or disproportionation.

### Example C—Embodiments of Particles Incorporating Therapeutics Therein Synthesized Via CDP

CDP is performed in a retrofitted CVD reactor with equipment as used in Example A, and corresponding to that depicted in FIG. 2.

Prior to CDP, the substrate was coated with a perflouorinated polymer (a “base layer”) resulting in an omniphobic coating that leads to sufficient contact angles for dropwise condensation during CDP. (The contact angle is evident, for example, in FIG. 8, which is a contact angle goniometry image of 2-hydroxyethyl methacrylate on the base layer.)

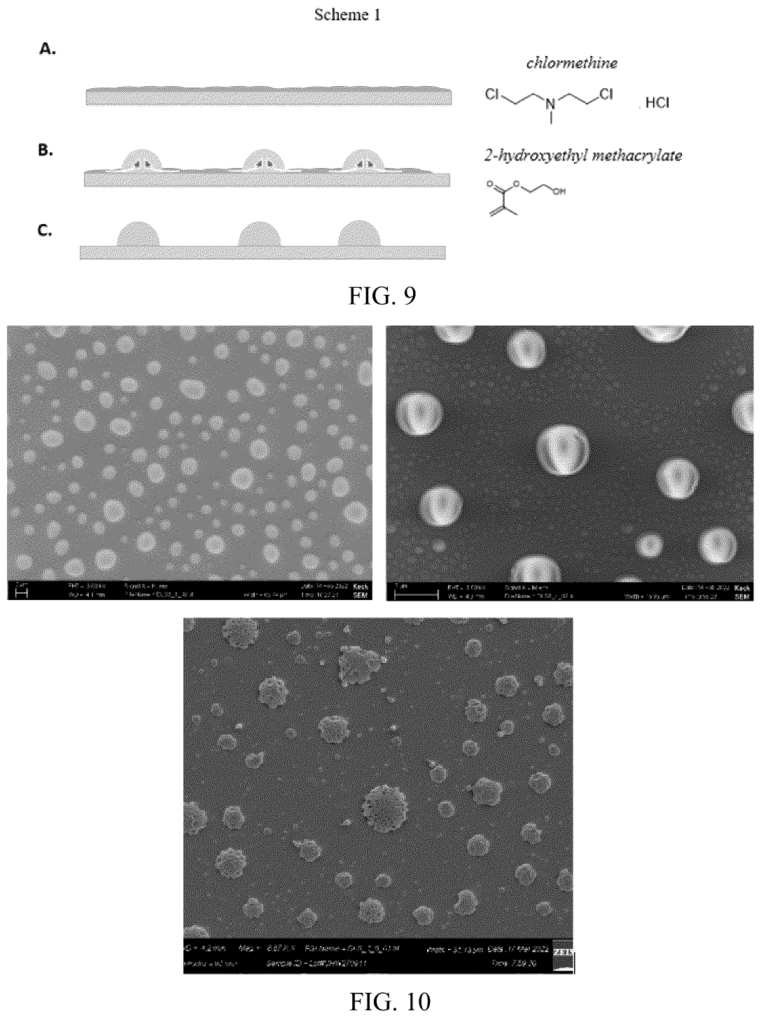

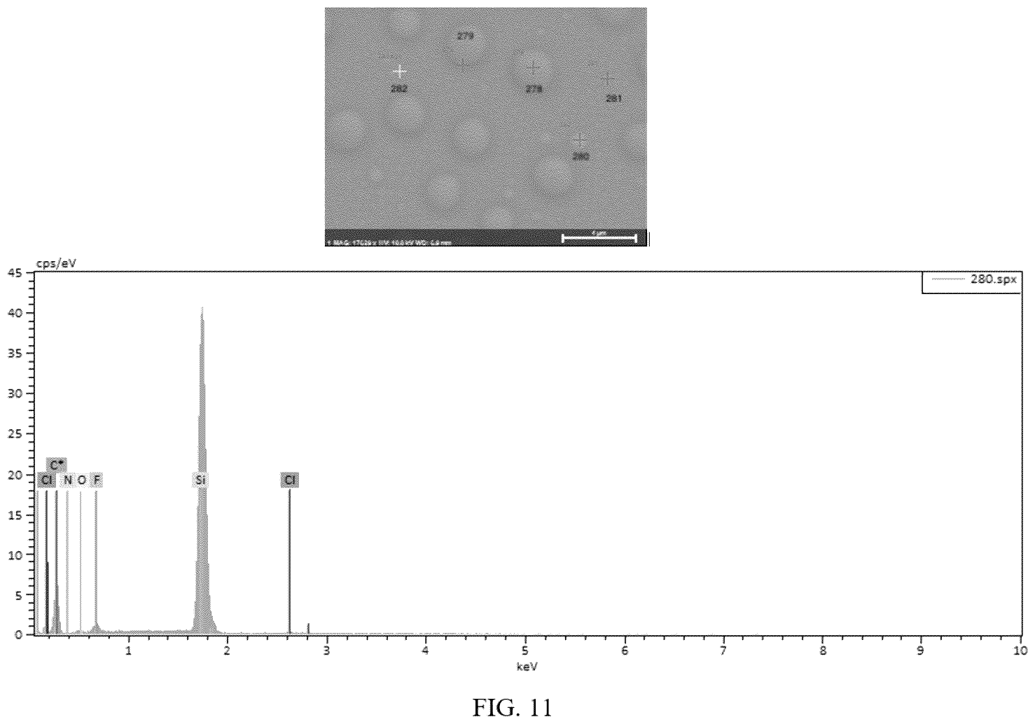

Chlormethine-containing particles were prepared according to Scheme 1, as shown in FIG. 9. (A) First, vapors of an additional agent (chlormethine) were condensed or solidified onto a cooled substrate. (B) Then, vapors of reagent comprising a monomer (2-hydroxyethyl methacrylate) were condensed onto the same surface, generating droplets in which the chlormethine dissolved (in other embodiments, the monomer may be delivered before or concurrently with the therapeutic agent). The vapors were introduced into the reactor chamber until saturation occurred at the substrate surface (this was monitored by a digital microscope). (C) Then, a vapor phase initiator was delivered to the chamber that was activated to radical form by the heated filament over the stage and contact with the droplets initiated polymerization that solidified the particles in place in which the therapeutic agent remains. FIG. 10 shows SEM images of resultant particles of poly(2-hydroxyethyl methacrylate) containing the chemotherapy drug chlormethine.

This example demonstrates the successful adaption of CDP to achieve the first ever vapor-phase incorporation of a therapeutic monomer into a polymer particle. The chemotherapeutic drug chlormethine was sublimated into the reactor and deposited onto the cooled base layer. When HEMA monomers were then condensed, chlormethine dissolved into the droplet. Polymerizing the HEMA generated a solid polymer particle containing chlormethine that could be released by the swelling action of this hydrogel polymer when wetted. SEM images confirmed the dome shape is preserved on the perfluorinated base layer even with the additional step to include the therapeutic molecule.

The altered CDP protocol adds only minutes to the procedure and offers a new approach to incorporating therapeutic agents by vapor. By avoiding the liquid environments of traditional polymerization techniques, CDP removes the requirement that a therapeutic agent is also soluble in the solution in which those syntheses are performed. Additionally, no purification steps are needed which improves both efficiency and safety in the process.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), “contain” (and any form contain, such as “contains” and “containing”), and any other grammatical variant thereof, are open-ended linking verbs. As a result, a method or device, composition, etc. that “comprises”, “has”, “includes” or “contains” one or more steps or elements possesses those one or more steps or elements, but is not limited to possessing only those one or more steps or elements. Likewise, a step of a method or an element of a composition or article that “comprises”, “has”, “includes” or “contains” one or more features possesses those one or more features, but is not limited to possessing only those one or more features.

As used herein, the terms “comprising,” “has,” “including,” “containing,” and other grammatical variants thereof encompass the terms “consisting of” and “consisting essentially of.”

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

All publications cited in this specification are herein incorporated by reference as if each individual publication were specifically and individually indicated to be incorporated by reference herein as though fully set forth.

Subject matter incorporated by reference is not considered to be an alternative to any claim limitations, unless otherwise explicitly indicated.

Where one or more ranges are referred to throughout this specification, each range is intended to be a shorthand format for presenting information, where the range is understood to encompass each discrete point within the range as if the same were fully set forth herein.

1. A method of synthesizing polymer particles, said method comprising:
introducing vapor-phase reagents into a reactor having a substrate;
forming condensed droplets of the reagents on the substrate;
initiating polymerization (e.g., by introducing polymerization initiator into the reactor or via photoinitiation); and
polymerizing the condensed droplets of the reagents,
thereby forming polymer particles.

2. The method according to claim 1, wherein the vapor-phase reagents include one or more monomers.

3. The method according to claim 2, wherein the monomers comprise 2-hydroxyethyl methacrylate, divinylbenzene, 4-vinylpyridine, benzyl methacrylate, ethylene glycol dimethacrylate, 1-vinylimidazole, cyclohexyl methacrylate, 2-(dimethylamino)ethyl methacrylate, or glycidyl methacrylate, or any combination thereof.

4. The method according to claim 1, wherein the polymerization initiator generates a radical that initiates a free radical polymerization upon contacting the condensed droplets.

5. The method according to claim 4, wherein the radical is generated by contacting the polymerization initiator with a heated filament array.

6. The method according to claim 1, further comprising:
introducing an additional agent (e.g., a therapeutic agent, for example, an anti-cancer agent, such as chlormethine) into the reactor.

7. The method according to claim 6, wherein the method is performed in the following order:
introducing an additional agent into the reactor;
introducing vapor-phase reagents into a reactor having a substrate;
forming condensed droplets of the reagents on the substrate;
introducing polymerization initiator into the reactor; and
polymerizing the condensed droplets of the reagents.

8. The method according to claim 6, wherein said introducing an additional agent into the reactor comprises sublimating the additional agent into the reactor.

9. The method according to claim 6, wherein after the additional agent and vapor-phase reagents are introduced into the reactor, the additional agent disperses or dissolves within the condensed droplet of the reagents.

10. The method according to claim 6, wherein the additional agent comprises:
a therapeutic agent (for example, an anti-cancer agent, such as a chemotherapeutic agent, e.g., chlormethine, 5-fluorouracil, camustine, ifosfamide, thiotepa, etc.);
an anti-cancer agent (for example, curcumin, kaempferol, paclitaxel, resveratrol, silamarin, vincristine, etc.);
an antimicrobial agent (for example, artemisinim, caffeic acid, capsaicin, coumarin, eugenol, menthol, etc.);
an anti-inflammatory agent (for example, capsaicin, colchicine, curcumin, epigallocatechin-3-gallate, quercetin, resveratrol, etc.);
a neuroprotective agent (for example, bacoside A, bilobalide, curcumin, galantamine, ginsenosides, withaferin A);
an antioxidant agent (for example, curcumin, cyanidin, gingerol, Ginkgo biloba, glycyrrhizin, quercetin, etc.);
a cardiovascular protection agent (for example, berberine, curcumin, dihydrotanshinone, quercetin, resveratrol, etc.);
essential oils, metals (silver ions, copper ions), zinc oxide, graphene oxide or carbon nanotubes, photoactive compounds (titanium dioxide, benzophenone, MoS₂, MnO₂, zinc oxide, gold nanoparticles), eugenol, menthol, eucalyptol, capsaicin, polyphenols, etc.;
a fuel agent, for example, cerium oxide+H₂O₂, calcium carbonate+acid, catalysts (e.g., aluminum oxide, copper oxide, silver oxide, iron oxide, cobalt oxide), etc.; or
a diagnostic and/or imaging agent, for example, fluorescent molecules, iodine, barium, supramagnetic iron oxide, bismuth, gold, etc.,
or a combination thereof.

11. The method according to claim 1, wherein said polymerizing solidifies polymerized condensed droplets in place on the substrate as solid polymer particles.

12. The method according to claim 1, wherein the polymer particles are hemispherical in shape.

13. The method according to claim 1, wherein the polymerization initiator is tert-butyl peroxide vapor that contacts a heated filament array to generate tert-butoxyl radicals that initiate free radical polymerization.

14. The method according to claim 1, wherein, during said introducing vapor-phase reagents into a reactor, the substrate is cooled.

15. The method according to claim 1, wherein said introducing vapor-phase reagents into a reactor comprises introducing the reagents into an evacuated, isolated chamber of the reactor, said chamber housing the substrate.

16. The method according to claim 1, wherein the substrate is functionalized (e.g., has a functionalized coating thereon).

17. The method according to claim 16 wherein the substrate comprises a perfluorinated polymer or poly(divinyl benzene).

18. The method according to claim 16, wherein the substrate comprises an omniphobic coating that enables dropwise condensation on the substrate.

19. The method according to claim 1, wherein following said polymerizing, the polymer particles are dry due to the solvent free nature of the method.

20. The method according to claim 1, wherein following said polymerizing, the polymer particles have not been exposed to liquid or solvent during the method.

21. The method according to claim 1, wherein following said polymerizing, atoms from any coating present on the substrate are not present in the polymer particle.

22. The method according to claim 1, wherein said polymerizing is complete within less than 120 seconds (e.g., less than 60 seconds).

23. The method according to claim 1, wherein the polymer particles have a size of 0.01 nm to 1,000,000 nm.

24. A polymer particle prepared according to the method of claim 1, comprising:
polymer material; and
optionally, mixed with the polymer material, an additional agent (e.g., a therapeutic agent).

25. (canceled)

26. The polymer particle according to claim 24, wherein:
the therapeutic agent is homogeneously or heterogeneously dispersed within polymer material; or
the polymer material contains a predominant chain, and end groups of the predominant chain are a methyl group on one end and a monomer on another end, for example, wherein the monomer end groups are of formula: 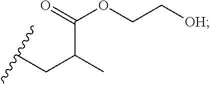 [SMILES: *CC(C)C(=O)OCCO]
or
the predominant chain does not comprise tertbutoxyl end groups.

27-29. (canceled)

30. The polymer particle according to claim 24, wherein the particle is prepared via CDP, and wherein:
the polymer material has a molecular weight higher than a corresponding polymer material prepared via iCVD (e.g., has a number averaged molecular weight (Mn) or a weight averaged molecular weight (Mw) greater than the corresponding polymer material prepared via iCVD; or
the polymer materials has longer polymer chains than a corresponding polymer material prepared via iCVD; or
the polymer materials has a lower polydispersity (PD) than a corresponding polymer material prepared via iCVD.

31. The polymer particle according to claim 30, wherein:
the polymer material is PHEMA; or
the particle does not comprise: a distinct outer coating containing the polymer material, the outer coating encapsulating an inner discrete particle; or
the particle is in the shape of a hemisphere.

32-33. (canceled)
